(19) United States
(12) Patent Application Publication
(10) Pub. No.: US 2011/0184427 A1
(43) Pub. Date: Jul. 28, 2011

(54) Oragienic Personal Dental Assistant

(76) Inventor: Ryan McClure, Mt. Pleasant, SC (US)

(21) Appl. No.: 12/621,334

(22) Filed: Jan. 28, 2010

(51) Int. Cl. A61B 17/24

(52) U.S. Cl. 606/106

(57) ABSTRACT

A hand-held suction device used to remove tonsilloliths, food, and other foreign matter from the mouth. Tonsilloliths form in the tonsil crypts or crevasses of the mouth and are a cause of chronic halitosis. Tonsilloliths generally become lodged in crevasses of the mouth and are unable to be removed without outside manipulation.

### CROSS-REFERENCE TO RELATED APPLICATIONS

Application Ser. No. 12/621,334 filing date: Jan. 28, 2010, non-provisional application

Application Ser. No. 12/621,255 filing date: Nov. 18, 2009, non-provisional application

Application Ser. No. 61/257,397 filing date: Nov. 2, 2009, provisional application

### STATEMENT REGARDING FEDERALLY SPONSORED RESEARCH OR DEVELOPMENT

N/A

### REFERENCE TO SEQUENCE LISTING, A TABLE, OR A COMPUTER PROGRAM LISTING COMPACT DISC APPENDIX

N/A

### BACKGROUND OF THE INVENTION

The oragienic personal dental assistant is an invention in the field of health and wellness, specifically, personal hygiene. The oragienic is personal, hand-held device that can be used to remove tonsilloliths and other matter and food particles from one's mouth. Tonsilloliths and left over food particles are associated with chronic halitosis. Removal of these items in conjunction with regular brushing, flossing and rinsing will help eliminate the causes of chronic halitosis. Currently, other methods used to remove these items include swabbing, irrigation, rinsing, or a visit to the dentist. None of these methods are as quick or as effective as the oragienic hand-held device. There is no other personal product like the oragienic personal dental assistant used to remove tonsilloliths on the market today.

### BRIEF SUMMARY OF THE INVENTION

The oragienic personal dental assistant is a hand-held, battery-operated suction device which can be used to clean one's mouth after a meal or any time the user requires its use. the oragienic will not cure, but will assist in the elimination of bad breath and will especially combat the sources of chronic halitosis. The oragienic personal dental assistant can be easily disassembled and stored in a small case that one may carry concealed on his person or in a carrying bag or briefcase. In addition to regular brushing, flossing, and general oral hygiene, the oragienic personal dental assistant will offer the missing link to help fully combat chronic halitosis.

### BRIEF DESCRIPTION OF THE SEVERAL VIEWS OF THE DRAWING

There are two drawings included with this application. The drawings are of the same view and are therefore numbered as 1/2 and 2/2:

1) Drawing 1/2 represents the oragienic personal dental assistant as a whole device separated into its several parts with no identifying information.

2) Drawing 2/2 represents the oragienic personal dental assistant as a whole device separated into its several parts with identifying information. Drawing 2/2 can be broken down into three separate parts:

A. Handle—the handle is made out of a hard plastic material and it contains the following features:

A.1) Battery housing to store two “AA” batteries, or two “AAA” batteries; a circuit board to conduct power; a moderately-powered mini-vacuum motor to create the suction needed to remove tonsilloliths and other foreign matter from the mouth; a light mounted on the interior and connected to the power source which will illuminate up through the handle, the reservoir head, and the straw; the light will be designed to automatically illuminate when the power button is depressed; A.2) the power button will be attached to the circuit card within the plastic casing and will mount on the outside of the handle; when depressed, this button will provide a complete circuit to power both the mini-vacuum motor as well as the light. At the end of the handle will reside a hole where the vacuum will be initiated. This end will incorporate an extension to which the detachable reservoir head will attach.

B. Detachable reservoir head—the detachable reservoir head will attach directly to the handle at one end. This end will create an airtight, vacuum-tight seal with the handle. The detachable reservoir head will be slightly hollowed out to allow for the flow of air created by the vacuum as well as the flow of tonsilloliths, and other food or foreign matter collected by the suction device. The detachable reservoir head will also incorporate a detachable reservoir (B.1) which can be easily detached for cleaning purposes. The reservoir can be securely screwed on and off. This reservoir specifically acts as a collection container for tonsilloliths, food, and any other foreign matter. After collection has taken place, the user will quickly understand the effect that the oragienic personal dental assistant has in combatting chronic halitosis when he/she is able to detach the reservoir and smell the contents to be rinsed away. At the other end of the detachable reservoir head, an extension will be incorporated to aid in the secure attachment of the cleaning straw or other attachments.

C. Detachable straw and other detachable accessories—the detachable straw and other accessories will be made from a softer plastic or silicone material. The accessories will securely lock in place to prevent detachment which may create a choking hazard. Once securely locked in place, the accessories will create the final airtight, vacuum-tight seal to create the suction needed to remove tonsilloliths, food, and other foreign matter from the mouth. The detachable straw and other accessories will be long enough to reach the tonsil crypts (crevices) near the back of the throat, but not long enough to reach the very back of the throat without the user forcing the device back that far. The device is not designed to reach the very back of the throat, but will be long enough to reach an imaginary line near the uvula and the tonsils. The design and material used for the detachable straw and other accessories is inspired by the same straws and accessories used by and deemed safe and effective by dentists all over. The straw and other accessories will be made of a soft plastic material, rubber or silicone and will be non-latex to avoid individual allergies to latex materials. The main attachment (the straw) will be equipped with a rubberized, or silicone head which is rounded and can be used to press against the tonsil area in the user's mouth to help free any lodged tonsilloliths, food, or any other foreign matter.

### DETAILED DESCRIPTION OF THE INVENTION

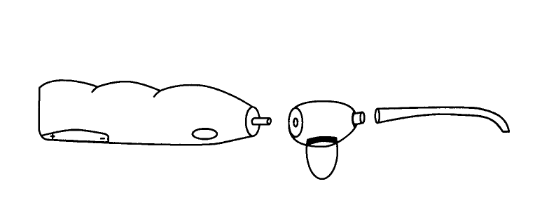

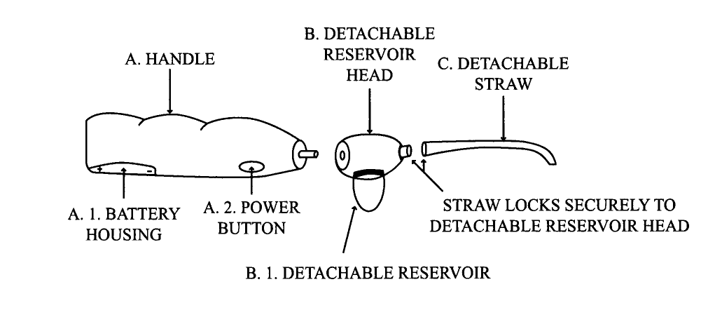

The oragienic personal dental assistant uses a mini-vacuum motor internally housed within a plastic casing to create a source for suction. The oragienic is battery-operated using either two “AA” batteries, or two “AAA” batteries as its source of power. The oragienic is hand-held and can be initiated by depressing a button (FIG. 2/2, A.2) on the plastic handle (FIG. 2/2, A.). The oragienic will only create suction with the button (FIG. 2/2, A.2) is depressed, so when the pressure is relieved from the button (FIG. 2/2, A.2), the suction ceases. The oragienic uses a straw raw (FIG. 2/2, C.) similar to that found in a dentist's office as well as other accessories that can easily be attached and detached. The accessories will attach to the handle (FIG. 2/2, A.) and reservoir head (FIG. 2/2, B.) and will lock in place so that the accessory does not detach and create a choking hazard. The oragienic is equipped with a reservoir (FIG. 2/2, B.1) to collect tonsilloliths, food, or any other foreign matter and it can be easily removed for cleaning.

The oragienic is also equipped with a bright light for illuminating the inside of one's mouth to search for tonsilloliths, food particles/pieces, and other foreign matter. While normal flushing of the mouth with water or with mouthwash and normal flossing helps to remove food particles and other foreign matter from one's mouth, there are other small pieces of food, matter, as well as tonsilloliths that lodge themselves in crevices (including the tonsily crypts) within the mouth which neither flossing nor flushing with water or mouthwash will remove. The oragienic helps to safely remove those smaller, hidden particles of food and other foreign matter. The oragienic will contain within its packaging directions and safety precautions for using this product. The directions will reveal the steps to remove tonsilloliths, food, and other foreign matter from those “secret” crevices (tonsil crypts) within the mouth. These locations and crevices trap food and when the food stays there for a significant time period, the food becomes a breeding ground for the bacteria that causes chronic halitosis. The smell that this bacteria creates goes well beyond the normal sulfurous smell of bad breath and can even smell similar to that of a septic tank.

The oragienic, in conjunction with normal brushing, flossing, and rinsing will help to eliminate the causes of chronic halitosis and bad breath in general. The oragienic will specialize in removing tonsilloliths. Tonsilloliths are calcareous matter which can consist of some combination of the following: dead white blood cells, oral bacteria, overactive salivary glands, mucus secretions, and residual of enzyme action on retained food. The tonsilloliths reside in the tonsil crypts or crevasses of the tonsils. In a medical study conducted in 2007, an association was discovered between tonsilloliths and bad breath with 75% of the subjects with tonsilloliths also had bad breath.

1. What I claim as my invention is the only personal, hand-held, oral suction device on the market used for the purpose of removing tonsilloliths from one's mouth.
